(19) United States
(12) Patent Application Publication
(10) Pub. No.: US 2015/0313630 A1
(43) Pub. Date: Nov. 5, 2015

(54) Solid Introducer Needle for Catheter

(71) Applicant: Optomeditech Oy, Espoo (FI)

(72) Inventors: Levon GASPARYAN, Helsinki (FI); Hans ROMBERG, Stutensee (DE)

(21) Appl. No.: 14/410,954

(22) PCT Filed: Aug. 21, 2012
(86) PCT No.: PCT/EP2012/066251

§ 371 Date: Jun. 25, 2015

(51) Int. Cl. A61B 17/34; A61M 25/01

(52) U.S. Cl. CPC A61B 17/3403; A61M 25/0105; A61M 2025/0166

(57) ABSTRACT

A solid introducer needle with a light guide unit for catheter assemblies is provided, which comprises a sharp rigid needle-like member and a light guide, forming a substantially monolithic structure. The monolithic structure is thus non-transparent for optical radiation over its entire length, allowing optical radiation to be emitted solely at the distal end thereof. Unit is preferably provided with an adapter, via which adapter unit may be connected to an electronic module, thus forming a catheter assembly. The light guide is configured to receive by its proximal end at least one light beam from at least one light source of the electronic module device, to further conduct said optical radiation beam throughout internal space thereof and to emit optical radiation beam at a distal end thereof. Optical radiation, thus emitted at the distal end of the unit, is preferably of such a wavelength, to be strongly absorbed by blood and walls of the blood vessels.

### FIELD OF THE INVENTION

The present invention relates to medical intravascular catheter systems and, in particular, to modified intravascular introducer needle for catheter assembly incorporating a light guide.

### BACKGROUND

Modern medical practice cannot be imagined without peripheral intravascular catheter systems. Conventional catheter systems commonly employ common medical needles comprising a lumen, although catheterization does not involve blood sample collection step. The introducer needle is intended basically for skin and/or blood vessel puncture; being withdrawn from the blood vessel after catheter itself is secured on patient's skin. Conventional method of placing the peripheral intravascular catheter into a blood vessel thus comprises skin puncture with an introducer needle, moving said needle in surrounding tissues forward towards target blood vessel, puncturing the wall of a target blood vessel and pushing a cannula of the catheter assembly inside target blood vessel while removing an introducer needle. A successful puncture of a blood vessel is confirmed by visual observation of blood flow into a flashback chamber of a catheter assembly. However, venipuncture has been traditionally associated with a number of common problems. One of those problems is difficulty in determination of the exact position of a needle tip inside the skin relatively to a blood vessel, especially on a dark skin. Another problem concerns the fact, that an observation of blood flow into a flashback chamber may not always serve a reliable indicator of penetration through a blood vessel wall. In case introducer needle penetrates blood vessel through and hits surrounding soft tissues, some blood would appear in the flashback chamber, thus giving a clinician a wrong indication of successful blood vessel entrance. Abovesaid problems become particularly important when placing a catheter system on any patient with small, deep, faulty or damaged veins, and/or in cases of emergency, ambulance and in children's hospitals.

From the construction point of view, the closest prior art for the present disclosure may be represented by trocars for preforming biopsy and/or for removing fluids from the abdomen, as well as by Central Venous Catheters (CVC), inserted into a large vein in the neck, and/or Peripherally Inserted Central Catheters (PICC), inserted into a vein in the arm, rather than in the neck. However, said introducer needles are commonly provided with the bore for fluids to pass. In addition, both biopsy procedures and insertion of CVC are commonly performed while observing the needle advancing into body by ultrasound visualization means. Additional confirmation of CVC needle entrance into vein is received by observing (by eye) blood in syringe barrel.

For peripheral intravascular catheters system various means are developed in order to localize blood vessels, including those light guides/illumination means.

Thus, prior art catheter systems commonly employ an introducer needle, comprising a sharp, tubular, hollow from inside metallic member, further provided with a void chamber, wherein blood flow occurs upon successful vein penetration. However, in order to puncture skin and blood vessel one may not necessarily need the needle provided with a bore therein. Confirmation of the successful blood penetration whether that is the case, is to be performed by other means, than visual observation of blood flow inside the void chamber.

### SUMMARY OF THE INVENTION

The objective of the invention is to provide a novel concept for medical introducer needles, by implementing a solid introducer needle for the intravascular catheter assembly which major purpose is piercing, wherein said needle is constructed as a substantially solid member provided with a light guide integrated therein so, that a substantially monolithic structure is created. Introducer needle in accordance with some embodiments thus does not comprise inner space for blood to flow in. Monitoring of blood vessel penetration events is implemented by optical means. Introducer needle may thus be utilized for fast and accurate localization of blood vessels and for detection of an exact moment of intravascular penetration, in accordance with certain embodiments.

The objective is attained by various embodiments of solid introducer needle in accordance with the present invention.

In one aspect of the present invention, a solid introducer needle with a light guide unit for intravascular catheter assembly is provided, comprising a rigid needle-like member with an integrated light guide, in accordance with the embodiments of the invention.

In one embodiment a solid introducer needle with a light guide unit is provided, said unit comprising a sharp, rigid, substantially tubular metallic member cut from its distal end longitudinally along certain length, thus forming along that length a duct-like structure, into which duct a light guide is positioned. The light guide is thus arranged to fill the rest of the tubular metallic member (non-cut) from inside. Plastic cannula is positioned over a duct-like needle structure with a light guide so, that above mentioned elements are being wrapped into the cannula tubing element. For clarity purposes, the solid introducer needle of the catheter assembly in accordance with present embodiment will be referred in this disclosure as “semilunar”.

In another embodiment a solid introducer needle with a light guide unit is provided, said unit comprising a sharp, rigid metallic bradawl blade-like member, enclosed into a light guide element, further provided with a cannula. For clarity purposes, the solid introducer needle of this embodiment will be referred as “bradawl-needle” in this disclosure.

In further embodiment of the invention a solid introducer needle with a light guide unit is provided, wherein a light guide is arranged to form a non-withdrawable piece with the introducer needle, wherein the lightguide element is preferably manufactured by filling a hollow needle piece by plastic material, thus sealing the needle over its whole length.

In further, substantially additional embodiment, an introducer needle unit is provided sealed from distal and proximal ends thereof by abovementioned light guide element.

In further embodiment a solid introducer needle with a light guide unit is provided, said introducer needle unit is coupled to an adapter member, further connected to the electronic module, wherein an introducer needle unit is adapted to receive a plastic cannula in a common way.

In further embodiment a solid introducer needle with a light guide unit is provided, comprising a light guide configured to receive by its proximal end at least one optical radiation beam of at least one wavelength from at least one optical radiation source, to further conduct said optical radiation beam throughout internal space thereof and to emit said optical radiation beam at its distal end, wherein the light guide is configured to receive, conduct and emit an optical radiation beam of such a wavelength, that is strongly absorbed by blood and/or walls of the blood vessels.

In another aspect of the invention, a method for detection of an exact moment of the intravascular penetration and for safeguarding the blood vessel from being damaged from inside is provided.

In further aspect of the invention a method for confirming an intravascular penetration of cannula tubing into the blood vessel is provided.

In still another aspect of the invention, a method for placing a peripheral intravascular catheter into a blood vessel is provided.

The term “solid” refers is this disclosure to an introducer needle unit and/or introducer needle assembly provided as a substantially monolithic structure with a light guide integrated therein. Hollow interiors are thus absent from the solid introducer needle and/or introducer needle assembly providing no or negligible opportunity for fluids to flow in.

The term “peripheral intravascular catheter” refers in this disclosure to a catheter assembly placed into a peripheral blood vessel, i.e. blood vessel located not in the chest or abdomen and thus being most commonly accessed by intravascular methods.

The term “blood vessel” may in this disclosure be vastly equivalent to the term “vein”, since peripheral veins are the most common access point for intravascular methods. To those skilled in art, however, it must be clear, that the term “blood vessel” may also relate to arteries.

The term “optical radiation” refers in this disclosure to radiation comprising a part of the electromagnetic spectrum and including ultraviolet, visible and infrared light ranges. The term may be used within this disclosure as an equivalent to the term “light”.

The term “introducer needle assembly” refers in this disclosure to an entity comprising a metallic needle, often referred to an “introducer needle”, with a flashback chamber typically being provided as an internal space of the introducer needle connection hub.

The term “introducer needle” refers in this disclosure to a needle for catheter systems, i.e. intended only for placing a catheter into blood vessel. Medical needles, intended for supplying/withdrawal fluids into the body, are not covered by this term within this disclosure.

The term “cannula” refers is this disclosure to a plastic part of the catheter assembly comprising flexible cannula tubing and cannula connection hub. Cannula is normally advanced into blood vessel and secured therein for further medicinal proceedings. The term “cannula” may substantially equal with the term “catheter” within said disclosure.

Different embodiments of the present invention will become apparent by consideration of the detailed description and accompanying drawings.

### BRIEF DESCRIPTION OF THE DRAWINGS

FIG. 1A illustrates a conventional peripheral intravascular catheter assembly.

FIG. 1B illustrates an introducer needle assembly of a conventional peripheral intravascular catheter assembly.

FIG. 1C illustrates a cannula of a conventional peripheral intravascular catheter assembly.

FIG. 1D illustrates a distal end of the conventional introducer needle provided with cannula tubing.

FIG. 1E illustrates a conventional process for the introduction of peripheral intravascular catheter assembly into blood vessel.

FIG. 2A illustrates a solid introducer needle with a light guide unit, in accordance with some embodiment.

FIG. 2B illustrates a solid introducer needle with a light guide unit in accordance with some embodiment, said unit provided with the adapter and connected to electronic module.

FIG. 3 is an enlarged view of a solid introducer needle with a light guide unit in accordance with some embodiment, provided with the adapter and connected to electronic module.

FIG. 4A schematically illustrates a side view of a solid introducer needle with a light guide, referred in present disclosure as a “semilunar needle”, in accordance with some embodiment.

FIG. 4B schematically illustrates a crosscut section of a solid introducer needle with a light guide, referred in present disclosure as a “semilunar needle”.

FIG. 4C schematically illustrates a sectional crosscut of a solid introducer needle with a light guide, referred in present disclosure as a “semilunar needle” in slightly different configuration.

FIG. 5A-B illustrate benefits of semilunar introducer needle application in comparison to conventional introducer needles, in particular, concerning size of light guide to be integrated within the introducer needle.

FIG. 6A schematically illustrates a side view of a solid introducer needle with a light guide, referred in present disclosure as a “bradawl needle”, in accordance with some embodiment.

FIG. 6B schematically illustrates a sectional crosscut of a solid introducer needle with a light guide, referred in present disclosure as a “bradawl needle”.

FIG. 7A schematically illustrates a side view of a solid introducer needle with a light guide arranged to form a non-withdrawable piece with the introducer needle by filling a hollow needle piece by plastic material, for example.

FIGS. 7B and 7C schematically illustrate a side view of a solid introducer needle with the hollow needle unit, provided sealed from distal and proximal ends thereof by a light guide element.

FIGS. 8A and 8B illustrate a process of blood vessel puncturing by means an intravascular catheter assembly comprising a solid introducer needle with a light guide.

### DETAILED DESCRIPTION OF THE EMBODIMENTS

Detailed embodiments of the present invention are disclosed herein with the reference to accompanying drawings. The same reference characters are used throughout the drawings to refer the same members. Following citations are used for the members:

### PRIOR ART

101—intravascular catheter assembly;
111—introducer needle assembly;
121—cannula;
102—metallic introducer needle of the introducer needle assembly;
103—flexible cannula tubing;
104—insertion (distal) end of the introducer needle;
105—chamber (proximal) end of the introducer needle;
106—introducer needle hub;
107—flashback chamber of the introducer needle assembly formed by a needle hub
108—blood;
109—connection hub of the cannula.

### PRESENT DISCLOSURE

201—intravascular catheter assembly provided with an electronic module;
211—introducer needle with a light guide;
202—rigid needle-like member of introducer needle assembly;
202D—distal section of rigid needle-like member;
202P—proximal section of rigid needle-like member;
203—cannula tubing;
204—distal end (tip) of the introducer needle;
205—proximal end of the introducer needle;
209—adapter;
210—light guide;
212—introducer needle bore;
213—protective layer;
214—empty space created between inner walls of cannula tubing, introducer needle and
210—light guide;
301—electronic module;
312—optical radiation source;
314—optical radiation converging device;
501—light spot.

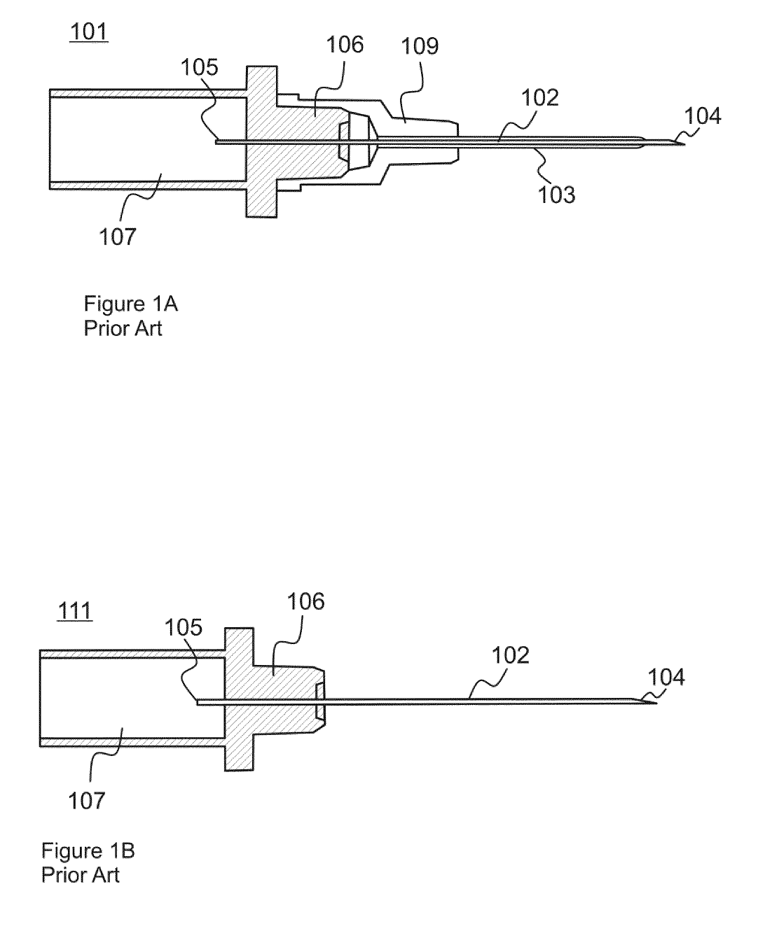

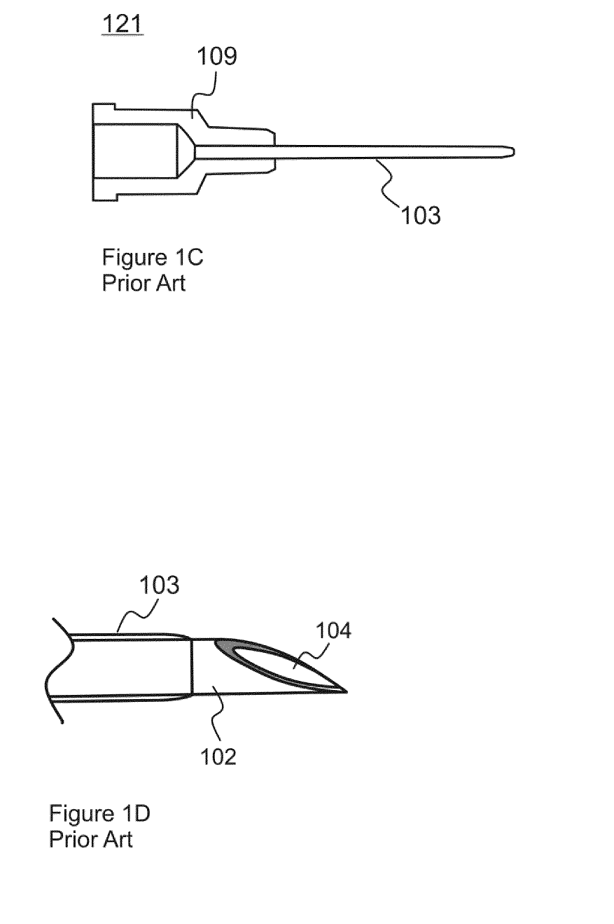

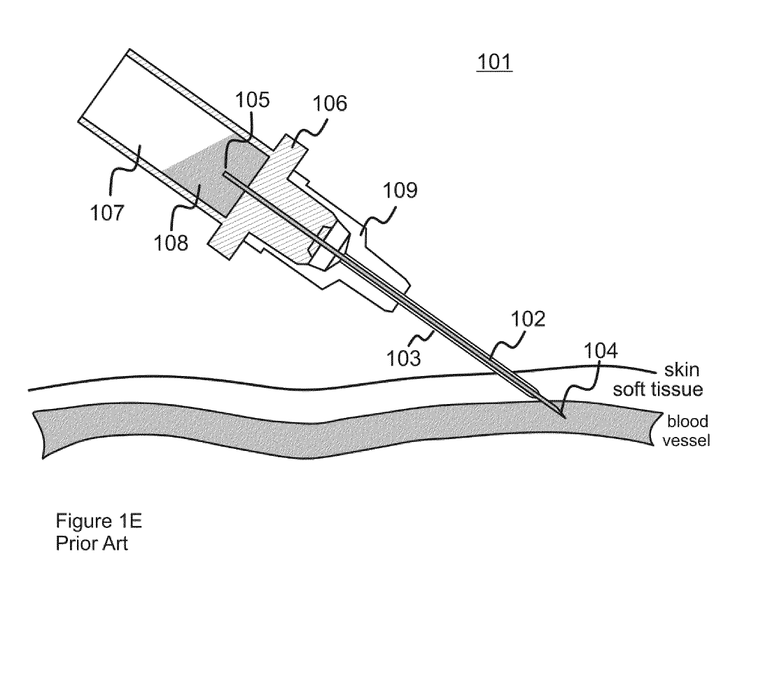

A conventional peripheral intravascular catheter assembly is illustrated by FIG. 1A (prior art). A catheter assembly 101 is shown (FIG. 1A), comprising an introducer needle assembly 111 (FIG. 1B) and a plastic cannula (FIG. 1C). The term “introducer needle assembly” refers herein to the metallic introducer needle 102 with a connection hub 106. A hollow interior of the connection hub 106 forms a flashback chamber 107. Onto a metallic introducer needle 102 an over-the-needle flexible plastic cannula 121 is mounted. Cannula 121 (FIG. 1C) thus comprises a cannula connection hub 109 and flexible cannula tubing 103. Cannula is a part of a catheter assembly that normally stays within blood vessel. Introducer needle 102 is provided with a sharp insertion end 104, referred herein as a distal end or the tip of the needle, and with a chamber end 105, referred herein as a proximal end.

Introducer needles for conventional catheter assemblies (like the one mentioned above) are normally provided as metallic, rigid, tubular and hollow from inside members, into the bore thereof blood flow occurs. Blood flows through the bore of the introducer needle 102, and via the chamber end 105 enters a flashback chamber 107. Visual monitoring of blood appearance in a flashback chamber 106 is a routine method to establish the fact of blood vessel penetration.

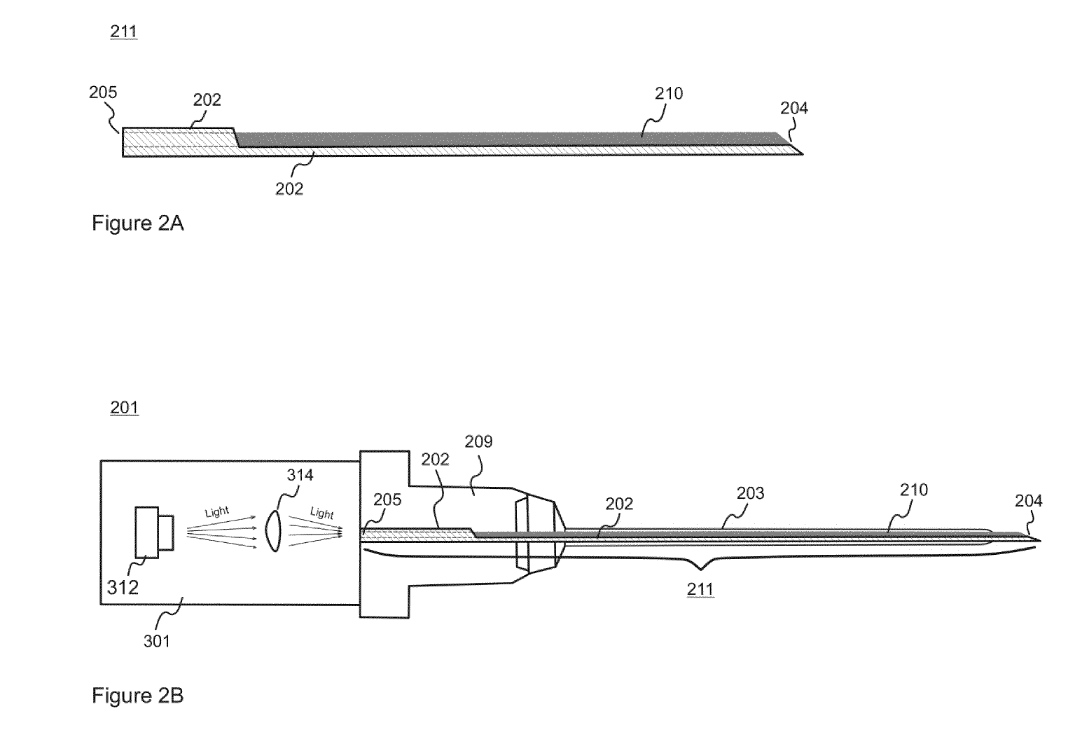

A solid introducer needle with a light guide, referred herein as unit 211, is thus provided (FIG. 2A), in accordance to some embodiment. Unit 211 is configured as a rigid needle-like member 202, supplied with a light guide 210, wherein light guide is preferably arranged to form a non-withdrawable (solid) structure with needle-like member 202. Needle-like member 202 may be metallic, as a conventional needle, however, any material of sufficient hardness and/or rigidity may be utilized for manufacturing said member, so far as the material is technically suitable for the purposes of the invention. Light guide 210 is preferably produced by the material with good light conducting properties and may be represented by an optical fiber permanently fixed inside the member 202, for example. Solid introducer needle with a light guide unit 211 may be releasably or permanently connected to the electronic module 301 by means of an adapter 209, thus forming an assembly structure 201. Such exemplary assembly structure 201 is represented on FIG. 2B. Assembly 201, however, may comprise unit 211, configured in accordance with any embodiment of present invention. Assembly structure may comprise, in addition, a cannula with tubing 203 with a connection means, realized as a connection hub, for example (not on the figure).

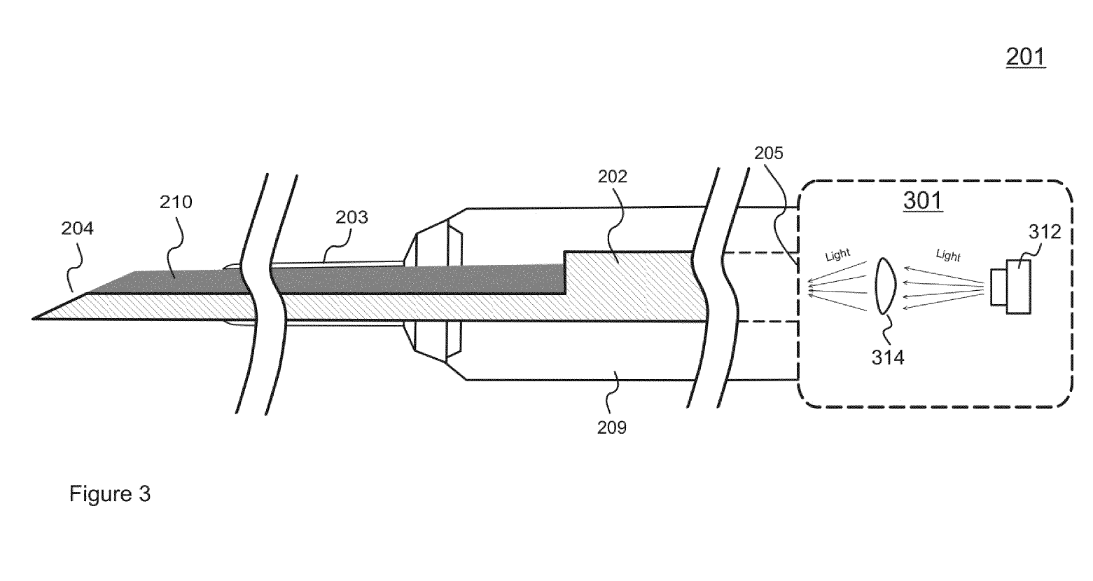

FIG. 3 is an enlarged view of the introducer needle assembly 201 of FIG. 2B. Introducer needle with a light guide unit 211 preferably integrated within the adapter 209; however 211 may be implemented as a withdrawable unit. Adapter 209 on the proximal side thereof comprises connection means, such as connection hub, for example, to be coupled to the electronic module 301. Electronic module 301 thus comprises at least one light source 312, light converging means 314, power source(s), such as battery(s) or the like, other electronic components and switches. Those are part of known prior art and are not described further in this document. Light source, however, may be arranged also inside the adapter 209, whether required for some particular applications.

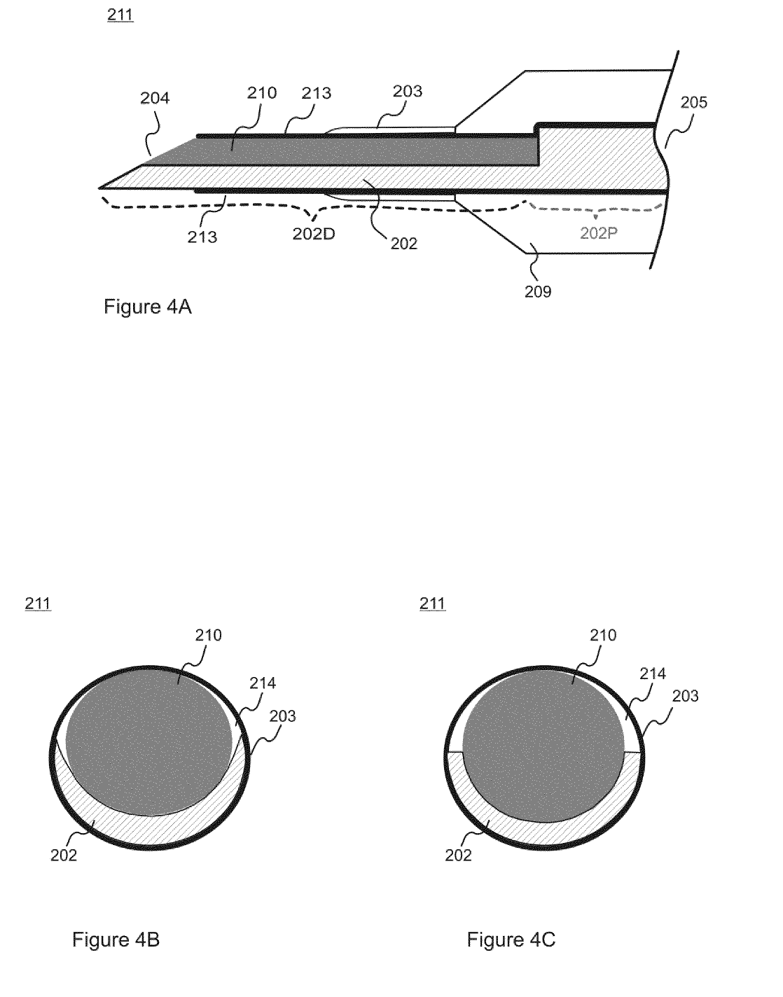

FIGS. 4A and 4B schematically illustrate a solid introducer needle with a light guide unit 211, referred herein as “semilunar”, in accordance with some embodiment. FIG. 4A provides a side view of the semilunar introducer needle unit 211 and FIG. 4B is a crosscut section. In accordance with the embodiment, unit 211 comprises a sharp, rigid, substantially tubular member 202, cut from its distal end longitudinally along certain length, thus providing a structure substantially comprised of two sections, distal 202D and proximal 202P (FIG. 4A). The proximal section 202P is provided as an elongated substantially tubular structure that may be best described as a pipe. The distal section 202D is provided in the form of a duct-like structure and may be best described as an above mentioned pipe, but horizontally severed. The distal end 204 (tip) of said distal section 202D is obliquely severed to form a sharp surface, suitable for puncturing skin, tissue and blood vessel. Member 202 is preferably metallic; however use of other material of sufficient hardness may not be ruled out. Preferably, the introducer needle with a light guide 211 is further surrounded by thin non-transparent protective film 213 (FIG. 4A). It is important to note, that light is intended to be emitted precisely at the distal end opening of the introducer needle with a light guide. Protective film 213 is thus intended to cover the introducer needle with a light guide unit 211 over its length in order to prevent light “leak” elsewhere than at the distal end 204.

By the proximal end 205 thereof introducer needle 201 may be connected to an electronic module 301 via an adapter 209. The adapter is positioned so, that an introducer needle proximal section 202P is completely hidden within the adapter, so as a part of a distal section 202D. The light guide 210 is integrated within needle-like member 202, being positioned along proximal and distal sections 202D and 202P to fill an entire proximal section from inside and to substantially fill the duct created by the distal section. Considering the fact, that proximal section 202P is hidden inside the adapter 209, the user may observe only horizontally cut duct-like distal section 202D with a light guide integrated therein. The cannula tubing 203 is positioned around the introducer needle with a light guide unit 211, ‘wrapping’ a part of the distal section 202D.

FIG. 4B illustrates a crosscut of the distal section 202D of the introducer needle with a light guide unit 201 of FIG. 4A with cannula tubing 203 wrapped over said unit. FIG. 4B shows a distinctive “semilunar” shape of the needle-like member 202, in accordance with some embodiment. FIG. 4B also schematically illustrates a light guide 210 position within distal section 202D of introducer needle unit 211. Light guide 210 is tightly positioned within the duct, provided by distal section 202D of unit 211, and fills up most of the space within tubular proximal section 202P. However, there may be still some empty space 214 left between light guide 210 and the walls of the tubular proximal section 202P of the unit 211, as well as between the light guide 210, inner walls of the duct-like distal section 202D of the unit 211 and inner surface of the cannula tubing 203 (FIG. 4B). However, those empty spaces (gaps) 214 are of such negligible size that no essential amount of fluid may flow in, therefore gaps 214 are not intended for receiving blood flow for further visual observations.

FIG. 4C illustrates a crosscut of the distal section 202D of the semilunar introducer needle with a light guide 211 in slightly different configuration. While a crosscut shape of the needle-like member 202 of FIG. 4B is literally “semilunar”, i.e. most closely reminds of a half-moon, the needle-like member 202 of FIG. 4C in crosscut thereof may be closer to a horizontally severed pipe, which severed walls in a crosscut are leveled to small plateaus and not sharpened, like in FIG. 4B.

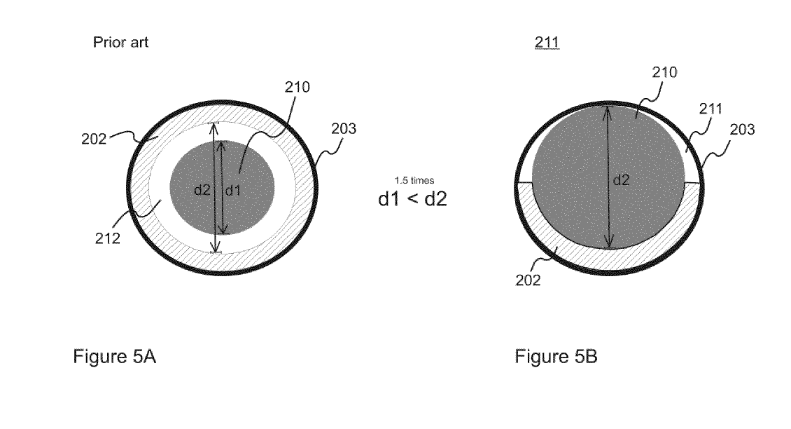

FIGS. 5A and 5B schematically illustrate the benefits of utilization of the introducer needle with a light guide 211 in substantially semilunar configuration, in accordance with above disclosed embodiments. As may be noticed from FIG. 5A, representing a conventional needle 202 with a light guide 210 within a bore 212 thereof, a diameter of the light guide (d1) that may be inserted into said needle is limited by the diameter of needle inner walls (d2). Taking into consideration the fact that needle 202 is supposed to provide space for fluid to flow in, the diameter of the light guide must be very small (FIG. 5A). However, utilization of semilunar configuration (FIG. 5B) provides an opportunity to utilize a light guide with wider diameter (d2), occupying practically a whole interior of the needle-like member 202 (FIG. 5B). The diameter of the light guide for semilunar configuration (FIG. 5B) may be 1.5 times wider than that for conventional configuration (FIG. 5A). Since ultrathin optical fibers may require a more powerful optical radiation source to emit same amount of optical radiation at distant end thereof as compared to wider optical fibers, the “semilunar” solution may be less expensive to manufacture.

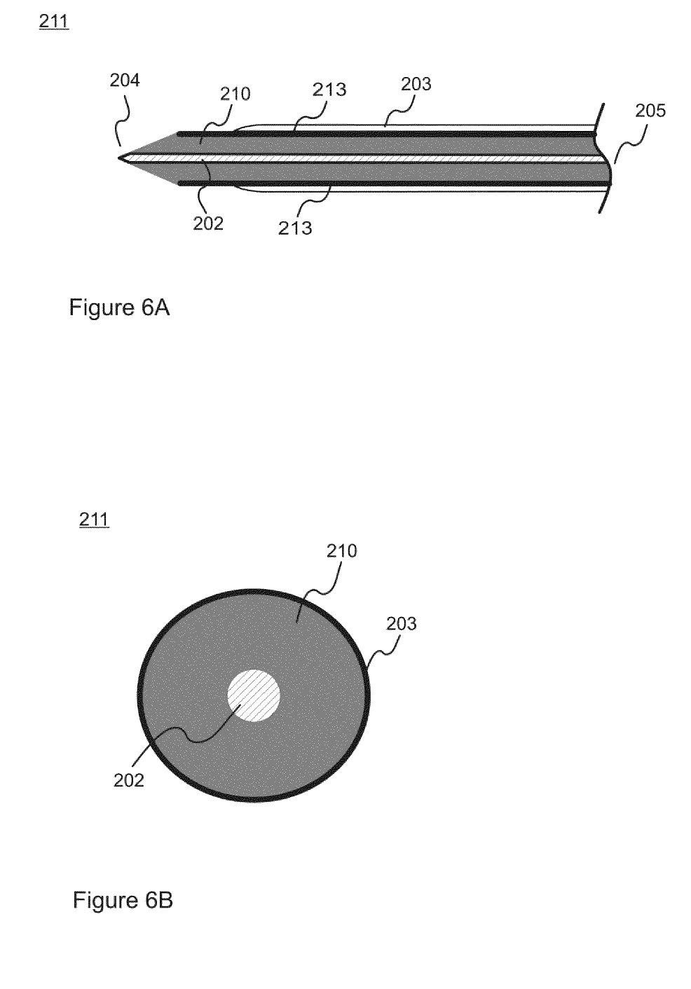

FIGS. 6A and 6B schematically illustrate another embodiment of solid introducer needle with a light guide unit 211, wherein the introducer needle is provided in so called “bradawl” configuration. FIG. 6A is a side view of the bradawl introducer needle with a light guide unit 211, and FIG. 6B is a crosscut section thereof. In accordance with this embodiment, a solid introducer needle is provided as a sharp, rigid, rod-like member 202 that may be best described as a bradawl blade-like member. Rod 202 may be metallic; however it may be manufactured from any other material of sufficient hardness, suitable for the purposes of the invention, such as hard transparent or non-transparent plastic or similar. Bradawl solid introducer needle 202 is enclosed into a light guide member 210 in a rod wire manner. The introducer needle of present embodiment may be best described as a common graphite pencil, said “pencil” comprises e.g. metallic rod in the middle, surrounded by the light-conductive layer. The tip 204 of the introducer needle with a light guide 211, in accordance with this embodiment, is sharpened in a bradawl blade-like manner (FIG. 6A). Bradawl solid introducer needle 202 rod may be provided retractable. Preferably, the light guide 210 is further surrounded by thin non-transparent protective film 213 (FIG. 6A). FIG. 6A also shows cannula tubing 203, placed over the introducer needle with a light guide 211. By the proximal end 205 thereof a bradawl introducer needle with a light guide may be connected to the adapter 209 and via an adapter—to the electronic module 301, like shown on FIG. 2B.

FIG. 4B illustrates a cross-cut of the bradawl introducer needle with a light guide unit 211 of FIG. 4A with cannula tubing 203 wrapped over it. The protective film 213 is not shown.

Returning further to FIG. 2B, both introducer needle with a light guide in bot semilunar and bradawl configurations, in accordance with above disclosed embodiments, may be connected to the electronic module 301. The connection is implemented via an adapter 209, which partly encloses an introducer needle with a light guide at proximal section thereof. Preferably, the introducer needle with a light guide unit 211 is manufactured as an assembly with the adapter 209, which further may be mounted onto an electronic module 301, or connected thereto by other suitable means. Said connection may be either releasable or permanent. Correspondingly, the manufacture of disposable, vacuum-packed assemblies 211+209 or 211+209+301 is possible; however the latter may be more cost-expensive.

Electronic module 301 may be therefore provided preferably as a multi-use unit, which comprises at least one optical radiation source, such as a light source 312, for example, and light converging means, such as lens 314, optical fiber or similar. Light source 312 is preferably laser source; however, any other suitable light source may be implemented herein. Electronic module 301 may be preferably equipped with an on-off switch, implemented, for example, as a manually operated pushbutton switch, a lever-actuated switch, a rotational switch, a slide switch with a seesaw action or any other suitable type of an on-off mechanism capable of providing a control over an on-off state of the electronic module. Electronic module may be equipped with a mechanism to control an amount of emitted light and/or color thereof. Electronic module 301 preferably comprises a power source, such as a battery, generator or the like. Electronic module 301 may comprise additional electronic components.

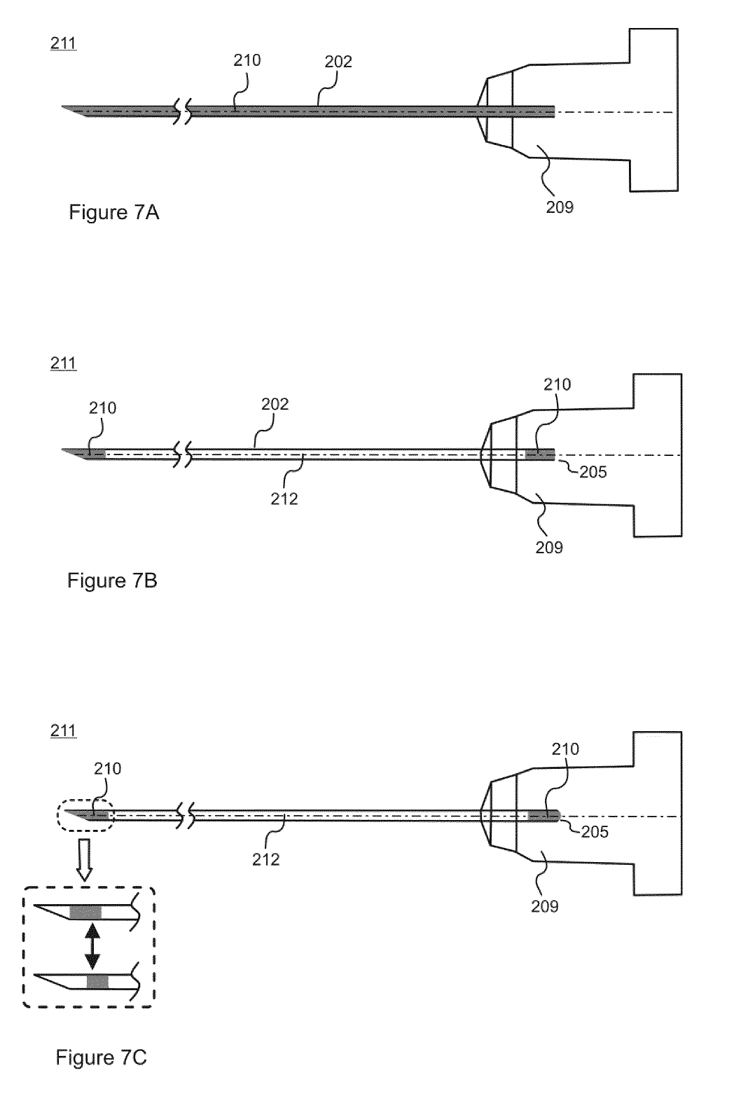

FIGS. 7A-C illustrates another embodiment of solid introducer needle with a light guide. The introducer needle with a light guide is shown on FIG. 7A-C coupled to the adapter 209. In accordance to this embodiment, light guide 210 (shown in dark) is arranged to form a non-withdrawable piece with an introducer needle 202 thus forming a monolithic structure with the introducer needle 202. Configuration of FIG. 7A is obtained by filling a bore of common tubular hollow metallic introducer needle 202 by light transmitting plastic material, serving herein as a light guide 210. The bore of the needle 202 thus becomes completely sealed over its whole length so, that no fluid may enter. FIGS. 7B and 7C illustrate a cost-effective version of same embodiment, wherein the introducer needle 202 is sealed only by its distal and proximal ends, while the bore 212 thereof remains empty. Polished inner surface of an introducer needle 202, however, may in this case operate as a light transmitting surface. Light beam is represented by a dashed line inside an introducer needle 202. Proximal end of the light guide, corresponding in FIG. 7B-C to a proximal end 205 of an introducer needle 202, may be simply cut (FIG. 7B), or be adapted to take a substantially spherical form (FIG. 7C) by mounting e.g. lens into proximal end of the needle, in order to enhance optical radiation input. Distal end ‘seal’ may be arranged into a very close proximity to a needle tip opening or alternatively at some distance from a needle tip opening (dashed box, FIG. 7C.

Light guide 210, in accordance to all embodiments, is therefore adapted to receive light originating from at least light source 312, to transmit light throughout its internal space and to emit at a distal end thereof. Light source may thus be arranged inside an electronic module 301, or inside an adapter 209. Whether light source is arranged within the adapter 209, electronic module may comprise a power source, such as battery.

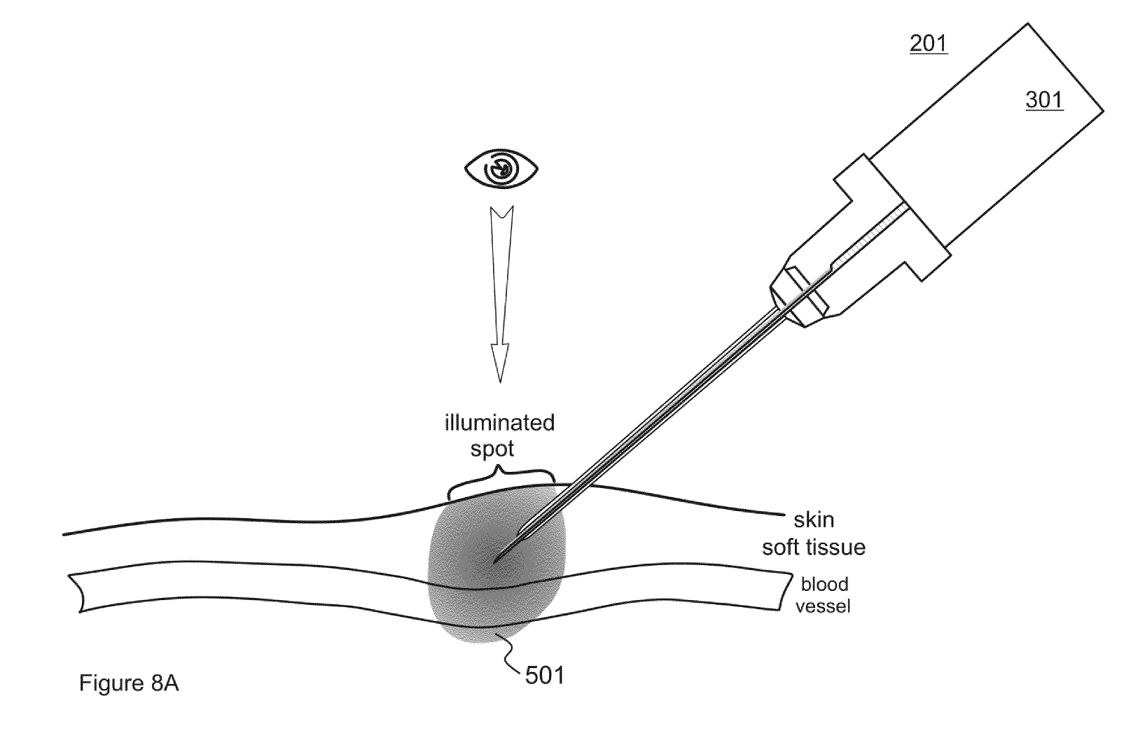

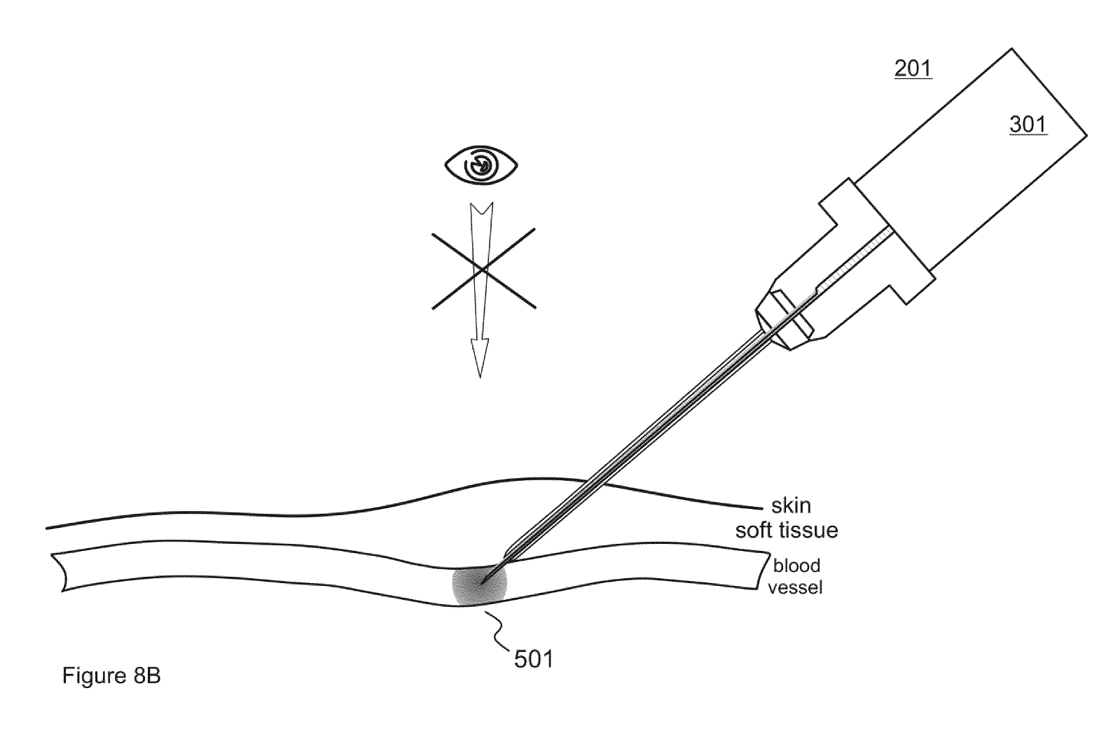

The process of entering a blood vessel by means of a catheter assembly 201 provided with the introducer needle with a light guide in accordance to above said embodiments, is illustrated by FIGS. 8A and 8B, wherein said needle is coupled to the electronic module 301 via an adapter 209. A light source is activated. Whether light of predetermined wavelength travels along light guide, a spot of light appears at a distal end 204 of the introducer needle unit 211. Preferably, at least one light source is adjusted to emit light of such wavelength, that is strongly absorbed by blood, in particular by red blood cells, and/or by the walls of blood vessels (such as veins and arteries), but is relatively weakly absorbed by skin, fat and other surrounding tissues. In order to enter a blood vessel an introducer needle with a light guide unit 211 has to penetrate tissues with different light scattering properties, such as skin, soft tissues and a wall of a blood vessel. When the distal end 204 of the introducer needle with a light guide unit 211 has already penetrated skin and tissues but has not yet entered a blood vessel, light illuminates surrounding tissues, being partly reflected and scattered therefrom, thus generating an illuminated spot on skin surface, that may be observed by eye. As soon as the introducer needle with a light guide unit 211 had entered blood vessel, light becomes absorbed by blood and/or the walls of a blood vessel, therefore causing the light spot to disappear from the skin surface. Since the penetration of a blood vessel wall by the needle tip is a very rapid process, optical properties of needle tip surrounding environment change momentarily from relatively low light absorption level to very high light absorption level. As a result, an outside observer (eye pictogram, FIG. 8A,B) may easily visually detect the moment when light, otherwise visible on skin, disappears; which event is indicative of a successful venipuncture. Since catheterization procedure does not require blood sample collection, and the necessity of observing blood appearing in the flashback chamber is now eliminated, solid introducer needle with an integrated light guide unit 211 in accordance with the embodiments of the invention may be successfully utilized for fast and accurate intravascular catheter placement.

Above described phenomenon may be observed upon selecting light of a specific wavelength, such as green, for example. However, light of other wavelengths of visible spectra, such as white, yellow and red, for example, may be utilized. In addition, IR radiation may be utilized as well.

The electronic module 301 may be, in addition, adapted to generate optical radiation, herein light, of at least two wavelengths at the same time by means of separate optical radiation sources. At least one optical radiation source is therefore configured to emit light of such a wavelength that is strongly absorbed by blood and the walls of blood vessels, in accordance with above disclosed.

It is to be understood, that above said optical radiation color spectra examples are not intended to limit the purposes of the invention, and the light source(s) provided with the electronic module 301 may be set up to generate optical radiation of any other suitable wavelength as far as the aforementioned effect from utilizing of the intravascular catheter assembly of the invention is achieved.

In another aspect of the invention, a method for detection of an exact moment of the intravascular penetration and for safeguarding blood vessel from being damaged from inside is provided, wherein said method is performed by means of the introducer needle with a light guide unit 211 in accordance with above said embodiments, and wherein said method comprises at least several of the following steps:
a. obtaining a catheter assembly 201 provided with solid introducer needle with a light guide unit 211, a cannula and an electronic module 301;
b. activating optical radiation source(s), said optical radiation source(s) implemented preferably in the form of laser source(s), for optical radiation to be observed on the distal end 204 of the introducer needle with a light guide unit 211;
c. puncturing the skin with by means of the introducer needle with a light guide unit 211;
d. localizing a blood vessel position intracutaneously by monitoring illumination events at the distal end 204 of unit 211;
e. detecting the moment when distal end of unit 211 penetrates the wall of a blood vessel and enters a lumen thereof by observing at least one of the following illumination events at the introducer needle distal end 204: instant disappearance of illumination, rapid illumination fading, rapid change in light color and/or rapid illumination fading along with rapid change in light color, wherein said events are dependent on the optical radiation wavelength utilized;
f. ensuring a correct position of the introducer needle with a light guide unit 211 within a lumen of a blood vessel by monitoring illumination events at the distal end 204 thereof;
g. advancing the cannula inside a lumen of a blood vessel.

In accordance with aforesaid aspect of the invention, said method may be applied equally efficiently to patients of any age group, independent of size, diameter and depth of blood vessels thereof.

In further aspect of the invention a method for confirming an intravascular penetration of cannula tubing into the blood vessel is provided, which method comprises at least several of the following steps:
a. obtaining a catheter assembly 201 provided with the introducer needle in accordance to some embodiments, and with the electronic module 301;
b. switching on the optical radiation source(s), said optical radiation source(s) implemented preferably in the form of laser source(s), of the electronic module 301 for optical radiation to be observed on the distal end 204 of the introducer needle 211;
c. puncturing the skin with the introducer needle 211;
d. localizing a blood vessel position intracutaneously by monitoring illumination events at the distal end 204 of the introducer needle 211;
e. detecting the moment when the introducer needle 211 penetrates the wall of a blood vessel and enters the lumen of a blood vessel by observing at least one of the following illumination events at the introducer needle distal end 204, said illumination events comprising disappearance of visible light and/or the instant change in visible light color;
f. ensuring a correct position of the introducer needle 211 inside the lumen of a blood vessel by monitoring illumination events at the introducer needle distal end 204;
g. observing blood flow inside the cannula tubing 203 from the distal end of the catheter assembly towards a proximal end thereof via space 214;
h. extending the cannula tubing 203 inside a lumen of a blood vessel.

In still another aspect of the invention, a method for placing a peripheral intravascular catheter into a blood vessel is provided in accordance with aforesaid embodiments, wherein said method comprises at least several of the following steps:
a. determination of a patient condition (child, adult, emergency, hospital etc) and determination of a blood vessel to be punctured condition (wide, thin, deep, damaged);
b. obtaining a catheter assembly 201 provided with solid introducer needle with a light guide unit 211, a cannula and an electronic module 301;
c. activating optical radiation source(s), said optical radiation source(s) implemented preferably in the form of laser source(s), for optical radiation to be observed on the distal end 204 of the introducer needle with a light guide unit 211;
d. puncturing the skin with by means of the introducer needle with a light guide unit 211;
e. localizing a blood vessel position intracutaneously by monitoring illumination events at the distal end 204 of unit 211;
f. detecting the moment when distal end of unit 211 penetrates the wall of a blood vessel and enters a lumen thereof by observing at least one of the following illumination events at the introducer needle distal end 204: instant disappearance of illumination, rapid illumination fading, rapid change in light color and/or rapid illumination fading along with rapid change in light color, wherein said events are dependent on the optical radiation wavelength utilized;
g. ensuring a correct position of the introducer needle with a light guide unit 211 within a lumen of a blood vessel by monitoring illumination events at the distal end 204 thereof;
h. advancing the cannula inside a lumen of a blood vessel;
i. withdrawing the introducer needle with a light guide unit 211 from a blood vessel, leaving the cannula inside a blood vessel;
j. securing the cannula over the skin of a patient and connecting an appropriate system thereto.

Important aspects of the invention are summarized in the following sentences:
1. An introducer needle 211 comprising a sharp rigid metallic member 202 provided with at least one light guide 210; wherein said introducer needle 211 is solid structure; wherein a light guide 210 is configured to receive by its proximal end at least one optical radiation beam of at least one wavelength from at least one optical radiation source of the electronic module device 301, to further conduct said optical radiation beam throughout internal space thereof and to emit said optical radiation beam at its distal end; and wherein a light guide 210 is configured to receive, conduct and emit an optical radiation beam of such a wavelength, that is strongly absorbed by blood and walls of the blood vessels and is reflected and/or scattered from blood vessel surrounding tissues.
2. The solid introducer needle 211 of sentence 1, wherein the needle member 202 is provided as a sharp, rigid, substantially tubular metallic member cut from its distal end longitudinally along certain length, providing a structure substantially comprised of two sections; wherein the distal section 202D is provided in the form of a duct-like structure, and the proximal section 202P is provided as an elongated substantially tubular structure; and wherein the light guide 210 is positioned along both proximal and distal sections, said light guide is arranged to fill an interior of a tubular proximal section 202P and further to be tightly positioned within the duct created by the distal section 202D.
3. The solid introducer needle 211 of sentences 1 and 2 arranged into a catheter assembly 201, said assembly comprising:
   a. the solid introducer needle 211 with a light guide 210, in accordance with sentences 1 and 2;
   b. a cannula provided with a tubing 203 and connection means;
   c. an adapter 209 and
   d. an electronic module 301,
   wherein the cannula tubing 203 is arranged to surround the structure formed by distal and proximal sections of the introducer needle with a light guide unit 211.
4. The solid introducer needle 211 of sentence 1, wherein the needle member 202 is provided as a sharp, rigid, rod-like member, substantially enclosed into a light guide element 210, which is further surrounded by a thin protective film 213, and wherein the tip 204 of the introducer needle 211 is sharpened in a bradawl blade-like manner.
5. The solid introducer needle 211 of sentences 1 and 4 arranged into a catheter assembly 201, said assembly comprising:
   e. an introducer needle 211 with a light guide 210, in accordance with sentences 1 and 4;
   f. a cannula provided with a tubing 203 and connection means;
   g. an adapter 209 and
   h. an electronic module 301,
   wherein the cannula tubing 203 is arranged to surround the structure formed by the introducer needle with a light guide unit 211 of sentence 4.
6. The solid introducer needle 211 of sentence 1 provided as a rigid tubular hollow member 202, which interior is filled by a light transmitting plastic material serving as a light guide 210, wherein the light guide 210 is arranged to form a non-withdrawable piece with member 202.
7. The solid introducer needle 211 of sentences 1 and 6, wherein said unit is provided as a rigid tubular hollow member 202 sealed by a light transmitting material solely at distal and proximal ends thereof.
8. The solid introducer needle 211 of sentences 1, 6 and 7, arranged into a catheter assembly 201, said assembly comprising:
   a. a rigid tubular hollow needle-like member 202 with a light guide 210 provided as a substantially monolithic structure therewith, in accordance with sentences 1, 6 and 7;
   b. a cannula provided with a tubing 203 and connection means;
   c. an adapter 209 and
   d. an electronic module 301,
   wherein the cannula tubing 203 is arranged to surround the structure formed by the member 202 with an integrated light guide 210 of sentence 6 and 7
9. The solid introducer needle 211 of sentences 2 and 4, which is permanently coupled to the adapter 209.
10. The solid introducer needle 211 of sentences 2 and 4, which is releasably coupled to an adapter 209.
11. The solid introducer needle 211 of sentences 3 and 5, wherein connection of 211 to the electronic module 301 via the adapter 209 is permanent.
12. The solid introducer needle 211 of sentences 3 and 5, wherein connection of 211 to the electronic module 301 via the adapter 209 is releasable.
13. The solid introducer needle 211 of sentences 3 and 5 provided with the electronic module 301, wherein the electronic module 301 comprises at least one optical radiation source, optical radiation converging means and at least one power source, and wherein said optical radiation source is configured to generate an optical radiation beam of such a wavelength, that is strongly absorbed by blood and walls of the blood vessels, but is reflected and/or scattered from surrounding tissues.
14. The solid introducer needle 211 of any of the preceding sentences, wherein the light guide 210 is manufactured from a substantially optical radiation conductive material, preferably optical fiber.
15. The solid introducer needle 211 of any of the preceding sentences, wherein the light guide 210 is configured to receive an optical radiation from a light source.
16. The solid introducer needle 211 of any of the preceding sentences, wherein the light guide 210 is configured to receive an optical radiation from a laser source.
17. The solid introducer needle 211 of any of the preceding sentences, wherein the light guide is configured to receive an optical radiation from a light diode.
18. The solid introducer needle 211 of any of the preceding sentences, wherein the light guide 210 is arranged to receive at least two optical radiation beams of different wavelengths from at least two optical radiation sources at the same time, one of which optical radiation beams is of a wavelength, that is strongly absorbed by blood and walls of the blood vessels, but is reflected and/or scattered from the surrounding tissue.
19. The solid introducer needle 211 of any of the preceding sentences, wherein the light source 312 is arranged within the electronic module 301.
20. The solid introducer needle 211 of any of the preceding sentences, wherein the light source 312 is arranged within the adapter 209.
21. The introducer needle 211 of any of the preceding sentences, wherein the adapter 209 is manufactured from a material, substantially non-transparent for optical radiation.
22. A method for detection of an exact moment of the intravascular penetration and for safeguarding blood vessel from being damaged from inside is provided, said method comprises:
   a. obtaining a catheter assembly 201 provided with solid introducer needle 211, with a cannula and with an electronic module 301;
   b. activating optical radiation source(s), for optical radiation to be observed on the distal end 204 of the introducer needle 211;
   c. puncturing the skin by means of the introducer needle 211;
   d. localizing a blood vessel position intracutaneously by monitoring illumination events at the distal end 204 of the unit 211;
   e. detecting the moment when distal end of the unit 211 penetrates the wall of a blood vessel and enters a lumen thereof by observing at least one of the following illumination events at the introducer needle distal end 204: instant disappearance of illumination, rapid illumination fading, rapid change in light color and/or rapid illumination fading along with rapid change in light color, wherein said events are dependent on the optical radiation wavelength utilized;
   f. ensuring a correct position of the introducer needle with a light guide unit 211 within a lumen of a blood vessel by monitoring illumination events at the distal end 204 thereof;
   g. advancing the cannula inside a lumen of a blood vessel.
23. A method for placing a peripheral intravascular catheter into a blood vessel, said method comprises:
   a. visual or by palpation determination of the condition of a blood vessel to be punctured;
   b. obtaining a catheter assembly 201 provided with solid introducer needle with a light guide unit 211, a cannula and an electronic module 301;
   c. activating optical radiation source(s), for optical radiation to be observed on the distal end 204 of the introducer needle with a light guide unit 211;
   d. puncturing the skin with by means of the introducer needle with a light guide unit 211;
   e. localizing a blood vessel position intracutaneously by monitoring illumination events at the distal end 204 of the unit 211;
   f. detecting the moment when distal end of the unit 211 penetrates the wall of a blood vessel and enters a lumen thereof by observing at least one of the following illumination events at the introducer needle distal end 204: instant disappearance of illumination, rapid illumination fading, rapid change in light color and/or rapid illumination fading along with rapid change in light color, wherein said events are dependent on the optical radiation wavelength utilized;
   g. ensuring a correct position of the introducer needle with a light guide unit 211 within a lumen of a blood vessel by monitoring illumination events at the distal end 204 thereof;
   h. advancing the cannula inside a lumen of a blood vessel.
   i. withdrawing the he introducer needle with a light guide unit 211 from a blood vessel, leaving the cannula inside a blood vessel;
   j. securing the cannula over the skin of a patient and connecting an appropriate system thereto.
24. An intravascular catheter needle 211 comprising:
   a. a needle member 202 having a proximal end and a distal end,
   b. a light guide 210 extending along at least a portion of the needle member 202 and terminating at the distal end of the needle member,
   wherein the catheter needle is devoid of a lumen forming a channel that would be suitable for guiding blood from the distal end to the proximal end of the needle member.
25. The catheter needle of sentence 24, wherein the distal end of the needle member is sharpened.
26. The catheter needle of sentences 24 and 25, wherein the needle member is a rigid metallic one-piece structure.
27. The catheter needle of any of the preceding sentences 24-26, wherein the light guide 210 is an optical fiber.
28. The catheter needle of any of the preceding sentences 24-27, wherein the needle member 202 comprises a tubular portion, in which it contacts completely a circumferential surface of the light guide, and a non-tubular portion, in which the needle member contacts only a portion of the circumferential surface of the light guide.
29. The catheter needle of sentence 28, wherein the non-tubular portion has a cross-section that has a semi-lunar shape.
30. The catheter needle of sentence 28, wherein the light guide 210 has the shape of a cylinder, and wherein the non-tubular portion has a cross-section that has the shape of a ring segment, and wherein an inner diameter of the ring is at least substantially equal to an outer diameter of the cylinder.
31. The catheter needle of any of sentences 28 to 30, wherein the tubular portion adjoins the proximal end of the needle member and the non-tubular portions adjoins the distal end of the needle member.
32. The catheter needle of any of sentences 24 to 27, wherein the needle member 202 has along its entire length a tubular shape such that it contacts completely a circumferential surface of the light guide accommodated therein.
33. The catheter needle of any of the sentences 24 to 31, wherein at least a portion of the light guide 210 has a tubular shape such that it contacts completely a circumferential surface of the needle member accommodated therein.
34. An intravascular catheter assembly 201, comprising an intravascular catheter needle 211 of any of the preceding sentences 24-33 and a cannula tubing 203 that is configured to be slid over a proximal end of the catheter needle 211 so as to surround at least a portion thereof
35. The catheter assembly of sentence 34, comprising a light source 312, which is configured to emit light that is coupled into the light guide 210, and an electronic module 301 that is configured to power the light source.
36. The catheter assembly of sentence 35, wherein at least 40% of light that is emitted by the light source has a wavelength between 500 nm and 580 nm.
37. The catheter assembly of any of the sentences 34 to 36, comprising an adapter 209 to which a proximal end of the light guide 210 is fixed, wherein the adapter is releasably connected to the electronic module 301.
38. The catheter assembly of any of the sentences 34 to 37, comprising a first light source that is configured to emit light having a first color and a second light source that is configured to emit light having a second color that differs from the first color.
39. The catheter assembly of sentence 38, wherein the light having the first color is more strongly absorbed by blood and walls of the blood vessels than light having the second color.
40. A method for the detection of an exact moment of the intravascular penetration and for safeguarding the blood vessel from being damaged from inside is provided, said method comprises:
   a. obtaining the intravascular catheter needle 211, provided with the electronic module 301;
   b. switching on the optical radiation source(s), said optical radiation source(s) implemented preferably in the form of laser source(s), of the electronic module 301 for optical radiation to be observed on the distal end 204 of the catheter needle 211;
   c. puncturing the skin with the catheter needle 211;
   d. localizing a blood vessel position intracutaneously by monitoring illumination events at the distal end 204 of the catheter needle 211;
   e. detecting the moment when the catheter needle 211 penetrates the wall of a blood vessel and enters the lumen of a blood vessel by observing one of the following illumination events at the catheter needle distal end 204, said illumination events comprising disappearance of visible light and the instant change in visible light color;
   f. ensuring a correct position of the catheter needle 211 inside the lumen of a blood vessel by monitoring illumination events at the catheter needle distal end 204;
   g. extending the cannula tubing 203 inside the lumen of a blood vessel.
41. A method for placing a peripheral intravascular catheter into a blood vessel, said method comprises:
   a. visual or by palpation determination of the condition of a blood vessel to be punctured;
   b. obtaining the intravascular catheter needle 211, provided with the electronic module 301;
   c. switching on the optical radiation source(s), said optical radiation source(s) implemented preferably in the form of laser source(s), of the electronic module 301 for optical radiation to be observed on the distal end 204 of the catheter needle 211;
   d. puncturing the skin with the catheter needle 211;
   e. localizing a blood vessel position intracutaneously by monitoring illumination events at the distal end 204 of the catheter needle 211;
   f. detecting the moment when the catheter needle 211 penetrates the wall of a blood vessel and enters the lumen of a blood vessel by observing one of the following illumination events at the catheter needle distal end 204, said illumination events comprising disappearance of visible light and the instant change in visible light color;
   g. ensuring a correct position of the catheter needle 211 inside the lumen of a blood vessel by monitoring illumination events at the catheter needle distal end 204;
   h. extending the cannula tubing 203 inside the lumen of a blood vessel.
   i. withdrawing the catheter needle 211 from a blood vessel, leaving the cannula tubing 203 inside a blood vessel;
   j. securing the cannula tubing 203 over the skin of a patient and connecting an appropriate system thereto.

The above description of various embodiments of the introducer needle with a light guide unit is given by way of example, and not limitation. Configurations of said unit, in accordance with the embodiments disclosed herein, are illustrative and are intended to provide a representative basis for teaching one skilled in art to employ the present invention in various configurations in regards to its aspects within the scope of the protective claims.

1. An introducer needle comprising:
a. a needle member having a proximal end and a distal end,
b. a light guide positioned within and extending along at least a portion of the needle member, the light guide terminating at the distal end of the needle member,
wherein the light guide forms a monolithic structure with the needle member such that the light guide cannot be withdrawn from the needle member, and
wherein the introducer needle does not comprise an inner space that is configured to receive blood through the distal end of the needle member.

2. The introducer needle of claim 1, wherein the distal end of the needle member is sharpened.

3. The introducer needle of claim 1, wherein the needle member is a rigid one-piece structure.

4. The introducer needle of claim 3, wherein the needle member is manufactured from metal.

5. The introducer needle of claim 1, wherein the light guide is an optical fiber.

6. The introducer needle of claim 1, wherein the needle member comprises a tubular portion and a non-tubular portion, wherein the tubular portion of the needle member surrounds a circumferential surface of the light guide, and wherein the non-tubular portion of the needle member contacts only a portion of the circumferential surface of the light guide.

7. The introducer needle of claim 1, wherein the needle member comprises only a non-tubular portion, and wherein the non-tubular portion of the needle member contacts only a portion of a circumferential surface of the light guide.

8. The introducer needle of claim 7, wherein the non-tubular portion has a cross-section that has a substantially semi-lunar shape.

9. The introducer needle of claim 1, further comprising cannula tubing surrounding the needle member and the light guide, wherein inner walls of the cannula tubing cooperate with the introducer needle and the light guide to define an empty space to enable blood flow.

10. The introducer needle of claim 6, wherein the light guide has the shape of a cylinder, wherein the non-tubular portion has a cross-section that has the shape of a ring segment, and wherein an inner diameter of the ring is at least substantially equal to an outer diameter of the cylinder.

11. The introducer needle of claim 6, wherein the tubular portion adjoins the proximal end of the needle member and the non-tubular portion adjoins the distal end of the needle member.

12. The introducer needle of claim 1, wherein the needle member has along its entire length a tubular shape that liquid-tightly surrounds the light guide accommodated therein.

13. The introducer needle of claim 1, wherein at least a portion of the light guide has a tubular shape such that it contacts completely a circumferential surface of the needle member.

14. An intravascular catheter assembly, comprising:
an introducer needle comprising:
a needle member having a proximal end and a distal end,
a light guide positioned within and extending along at least a portion of the needle member, the light guide terminating at the distal end of the needle member,
wherein the light guide forms a monolithic structure with the needle member such that the light guide cannot be withdrawn from the needle member, and
wherein the introducer needle does not comprise an inner space that is configured to receive blood through the distal end of the needle member; and
a cannula tubing that is configured to slide over a proximal end of the introducer needle so as to surround at least a portion thereof.

15. The catheter assembly of claim 14, further comprising:
a first light source that is coupled to the light guide, wherein the first light source is configured to emit light into the light guide; and
an electronic module that is configured to power the light source.

16. The catheter assembly of claim 15, wherein at least 40% of light that is emitted by the first light source has a wavelength between 510 nm and 600 nm.

17. (canceled)

18. The catheter assembly of claim 15, further comprising a second light source coupled to the light guide, wherein the first light source is configured to emit light having a first color and the second light source is configured to emit light having a second color that differs from the first color.

19. The catheter assembly of claim 18, wherein the light having the first color is more strongly absorbed by blood and walls of blood vessels than light having the second color.

20. A method for detecting intravascular penetration within a patient, comprising:
a. using an intravascular catheter assembly, the intravascular catheter assembly comprising:
an introducer needle comprising:
a needle member having a proximal end and a distal end,
a light guide positioned within and extending along at least a portion of the needle member, the light guide terminating at the distal end of the needle member,
wherein the light guide forms a monolithic structure with the needle member such that the light guide cannot be withdrawn from the needle member, and
wherein the introducer needle does not comprise an inner space that is configured to receive blood through the distal end of the needle member; and
a cannula tubing that is configured to slide over a proximal end of the introducer needle so as to surround at least a portion thereof;
a first light source that is coupled to the light guide, wherein the first light source is configured to emit light into the light guide; and
an electronic module that is configured to power the first light source;
b. switching on the electronic module so that light from the first light source is observed at the distal end of the introducer needle;
c. puncturing the skin of the patient with the introducer needle;
d. localizing a blood vessel position intracutaneously by monitoring illumination events at the distal end of the introducer needle;
e. detecting when the introducer needle penetrates a wall of a blood vessel and enters a lumen of a blood vessel by observing at least one illumination event at the distal end of the introducer needle, wherein the at least one illumination event comprises at least one of disappearance of visible light or an instant change in visible light color;
f. ensuring a correct position of the introducer needle inside the lumen of the blood vessel by monitoring illumination events at the distal end of the introducer needle; and
g. extending the cannula tubing inside the lumen of the blood vessel.

21. The method of claim 20, wherein the cannula tubing surrounds the needle member and the light guide, wherein inner walls of the cannula tubing cooperate with the introducer needle and the light guide to define an empty space to enable blood flow, and wherein the method further comprises
observing blood flow inside the empty space of the cannula tubing from the distal end of the catheter assembly towards a proximal end thereof.

22. (canceled)

23. The introducer needle of claim 6, wherein the non-tubular portion has a cross-section that has a substantially semi-lunar shape.

24. The catheter assembly of claim 14, wherein the light guide of the introducer needle is an optical fiber.

25. The catheter assembly of claim 14, wherein the needle member of the introducer needle comprises a tubular portion and a non-tubular portion, wherein the tubular portion of the needle member surrounds a circumferential surface of the light guide, and wherein the non-tubular portion of the needle member contacts only a portion of the circumferential surface of the light guide.

26. The catheter assembly of claim 14, wherein the needle member of the introducer needle comprises only a non-tubular portion, and wherein the non-tubular portion of the needle member contacts only a portion of a circumferential surface of the light guide.
